(19) United States
(12) Patent Application Publication
(10) Pub. No.: US 2014/0220292 A1
(43) Pub. Date: Aug. 7, 2014

(54) SYSTEMS AND METHODS FOR ENHANCED PROTECTION DURING BLOOD TUBING SEALING

(71) Applicant: New York Blood Center, New York, NY (US)

(72) Inventor: Michael Smyth, Valley Stream, NY (US)

(73) Assignee: New York Blood Center, New York, NY (US)

(21) Appl. No.: 14/248,651

(22) Filed: Apr. 9, 2014

Related U.S. Application Data
(63) Continuation of application No. 13/096,918, filed on Apr. 28, 2011, now abandoned.
(60) Provisional application No. 61/356,985, filed on Jun. 21, 2010.

(51) Int. Cl. A61B 5/15

(52) U.S. Cl. CPC A61B 5/150351; A61B 5/150274
USPC 428/99; 29/428

(57) ABSTRACT

Disclosed herein are systems and methods for user protection from blood splatter during blood tubing sealing. Systems and methods described herein utilize a blood splatter safety shield attached to a handheld blood tubing sealer, comprising one or more barriers, wherein at least one barrier runs substantially perpendicular to the handheld blood tubing sealer separating blood tubing being sealed from a user of the handheld blood tubing sealer.

### CROSS REFERENCE TO RELATED APPLICATIONS

The present application is a continuation of U.S. patent application Ser. No. 13/096,918, filed Apr. 28, 2011, which claims the benefit under 35 U.S.C. §119(e) to U.S. Provisional Application Ser. No. 61/356,985 filed Jun. 21, 2010, the entire contents of all of which are hereby incorporated by reference in their entirety.

### FIELD OF INVENTION

This application claims priority to provisional application serial number 61/356,985, filed June 21, 2010, the disclosure of which is incorporated herein by reference in its entirety.

Systems and methods disclosed herein generally relate to user protection during the sealing of blood tubing. More particularly, the present disclosure relates to systems and methods to protect a phlebotomist or other user from blood or fluid splatter during blood tubing sealing with a safety shield attached to a handheld blood tubing sealer between the user and the blood tubing.

### BACKGROUND

After a blood unit is extracted from a patient or donor, blood tubing must be safely sealed. Blood tubing is tubing used in blood, plasma, platelet, blood component, and other bodily fluid collection. Sealing of blood tubing prevents exposure of users or other handlers to blood-borne pathogens (for example, Human Immunodeficiency Virus (HIV), Hepatitis B (HBV), Hepatitis C (HCV), syphilis, and malaria); allows for easier transportation, handling, identification, and testing of samples; and prevents contamination of such samples. Portable blood tubing sealers, such as but not limited to, a Fenwal Autosealer Tubing Sealer, a Fresenius Kabi Composeal Mobilea tube sealer, or a similar battery-powered tubing sealer, are regularly utilized for such sealing.

During the sealing process, blood tubing is at risk of rupturing. If a segment of blood tubing ruptures during sealing, the phlebotomist or other person involved in sealing the blood tubing can be splattered with blood, blood components, or other bodily fluids.

Because of the safety risks involved in exposure to blood and bodily fluids, phlebotomists and other users typically use face and/or eye protection during the sealing process. The face and/or eye protection currently used, including shields that are worn by individuals can be cumbersome and unwieldy. Such shields can obstruct a user's view and can be uncomfortable for a user to wear. In addition, face and/or eye shields need to be provided to each potential user or person involved during the blood sealing process. Accordingly, there is room for improvement in user protection during blood tubing sealing.

### SUMMARY OF THE INVENTION

The systems and methods disclosed herein are designed to shield a user from blood splatter or exposure to blood-borne pathogens during the sealing of blood tubing. The presently disclosed shields provide comfortable and safe protection from possible splatter during blood tubing sealing.

Particularly, one embodiment includes a blood splatter safety shield configured for attachment to a handheld blood tubing sealer comprising at least one barrier and an attachment mechanism to attach the at least one barrier to the handheld blood tubing sealer. In some embodiments, the handheld blood tubing sealer can comprise a seal slot and a seal lamp, and the blood splatter safety shield can be attached between the seal slot and the seal lamp of the handheld blood tubing sealer.

Embodiments of the present description can be attached to handheld blood tubing sealers that: are connected to a power source via a cord or cable; are standalone, self-contained units with internal power sources; or are standalone, self-contained sealers that can be charged on a charging station .

In some embodiments, when the blood splatter safety shield is attached to the handheld blood tubing sealer, the at least one barrier runs substantially perpendicular to the handheld blood tubing sealer between the blood tubing that is being sealed and a user of the handheld blood tubing sealer.

In another embodiment, the at least one barrier is substantially planar. In some embodiments, the at least one barrier is substantially transparent.

Certain blood splatter safety shields further comprise a second barrier. In some embodiments, the second barrier runs substantially parallel to the handheld blood tubing sealer and can be above the seal slot of the handheld blood tubing sealer when the blood splatter safety shield is attached to the handheld blood tubing sealer. In one embodiment, the second barrier is substantially planar. In other embodiments, the second barrier is substantially transparent.

The attachment mechanism of some embodiments of the blood splatter safety shield comprises an opening in the at least one barrier through which the handheld blood tubing sealer can pass and be secured in place. In another embodiment there is a gasket between the attachment mechanism and the handheld blood tubing sealer.

According to the present disclosure, another embodiment includes a system comprising a blood splatter safety shield and a handheld blood tubing sealer wherein the blood splatter safety shield is attached to the handheld blood tubing sealer and comprises at least one barrier. In some systems, the handheld blood tube sealer comprises a seal slot and a seal lamp, and the blood splatter safety shield is attached to the handheld blood tubing sealer between the seal slot and the seal lamp.

In some embodiments of the system, the blood splatter safety shield is removably or permanently attached to the handheld blood tubing sealer.

In one embodiment of the system, the splatter safety shield is attached to the handheld blood tubing sealer through an opening in the at least one barrier. Certain embodiments of the system of the present disclosure further comprise a gasket at the opening.

In another embodiment, during use, the at least one barrier runs substantially perpendicular to the handheld blood tubing sealer between blood tubing and a user of the handheld blood tubing sealer. In another embodiment, the at least one barrier is substantially planar and/or substantially transparent.

Another embodiment of this system further comprises a second barrier. In another embodiment the second barrier runs substantially parallel to the handheld blood tubing sealer and can be above the seal slot of the handheld blood tubing sealer. In another embodiment the second barrier is substantially planar and/or substantially transparent.

The present disclosure also includes methods. One method includes a method of preventing blood and/or fluid splatter from contacting a phlebotomist comprising attaching a blood splatter safety shield to a handheld blood tubing sealer.

Another method includes at least the steps of attaching a blood splatter safety shield of the present description to a handheld blood tubing sealer (such as, without limitation, a battery-powered tubing sealer), sealing blood tubing using a handheld blood tubing sealer, and collecting any blood splatter generated from the sealing process on the blood splatter safety shield.

### BRIEF DESCRIPTION OF THE DRAWINGS

FIG. 1 illustrates an exemplary embodiment of a blood splatter safety shield described herein attached to an exemplary handheld blood tubing sealer.

FIG. 2 is a side view of the exemplary embodiment depicted in FIG. 1.

FIG. 3 is a side view of an alternative exemplary embodiment of a blood splatter safety shield described herein attached to an exemplary handheld blood tubing sealer.

FIG. 4 illustrates yet another exemplary embodiment of a blood splatter safety shield described herein attached to a different exemplary handheld blood tubing sealer.

FIG. 5 illustrates a blood tubing sealer and its handheld blood tubing sealer to which an exemplary blood splatter safety shield of the present disclosure can be attached.

### DETAILED DESCRIPTION

After a blood unit has been extracted, blood tubing must be sealed to prevent exposure of users or other handlers to blood-borne pathogens, to allow for easier transportation, identification, handling, and testing of samples, to maintain sterile samples, and to prevent contamination of the same. However, the sealing process can result in blood tubing ruptures, potentially exposing phlebotomists, or others involved in the sealing process to dangerous pathogens.

Exemplary systems and methods of the present description utilize a blood splatter safety shield attached directly or indirectly to a handheld blood tubing sealer to protect users of the handheld blood tubing sealer from blood splatter and the associated pathogens. The blood splatter safety shields are a comfortable, portable, and inexpensive alternative to unwieldy face shields.

“Blood tubing” as used herein generally refers to tubing used in blood, plasma, platelet, blood component, and other bodily fluid collection. “Tube” and “tubing” are used interchangeably herein to refer to passageways between at least two reservoirs defined by a boundary. Blood tubing and the embodiments of the present disclosure have application in, without limitation, blood pack units, apheresis kits, plasma packs, and automated blood component collection kits.

“Blood” as used herein generally refers to blood, plasma, platelets, blood components, and other bodily fluids of humans and other blood-containing animals. Embodiments of the present disclosure are not limited to use in human blood and bodily fluid collection, but can also be used for veterinary purposes.

“Handheld blood tubing sealer” as used herein generally refers to any of: standalone, self-contained blood tubing sealers with an integrated power source including, without limitation, batteries or solar power; standalone, self-contained sealers that can be charged on a charging station; and hand units of blood tubing sealers that are connected to the blood tubing sealers via a cord or coaxial cable.

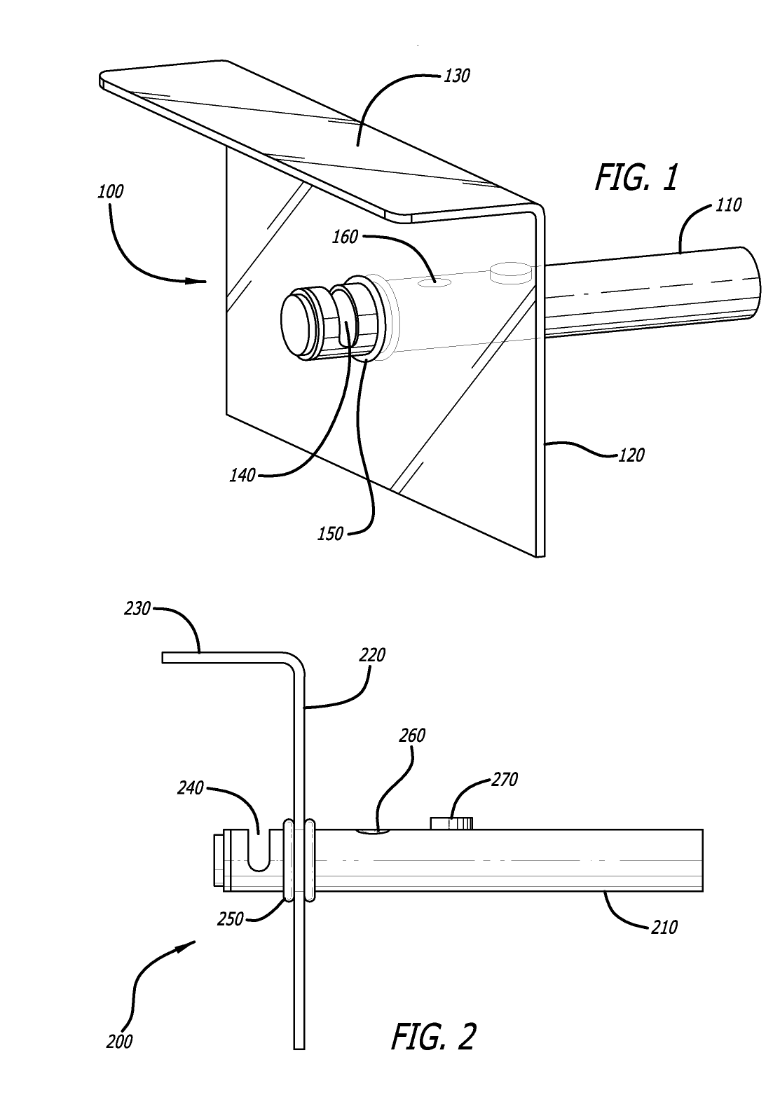

FIG. 1 illustrates an exemplary blood splatter safety shield 100 attached to an exemplary handheld blood tubing sealer 110. Blood splatter safety shield 100 comprises at least one barrier 120 and attachment mechanism 150. The at least one barrier 120 runs substantially perpendicular to handheld blood tubing sealer 110, such that it can separate a segment of blood tubing that is being or is sealed by handheld blood tubing sealer 110 from the phlebotomist or user of handheld blood tubing sealer 110. Accordingly, if blood tubing ruptures during sealing, blood splatter can be captured by at least one barrier 120.

The barriers described herein can be, without limitation, planar, curved, convex or concave. In fact, the barriers can take any shape sufficient to prevent blood or fluid splatter from reaching the user of a handheld blood tubing sealer. The barriers described herein can also be manufactured using any appropriate material. The material can be rigid or semi-rigid. The material must simply be sufficiently rigid to maintain a shape that prevents blood splatter from reaching the user of the handheld blood tubing sealer. In certain embodiments, the material/barriers are substantially transparent. In other embodiments, only a portion of the material/barriers are transparent. Other embodiments may be opaque with reflective surfaces attached or positioned nearby to allow the phlebotomist to see around the barrier.

Certain embodiments of blood splatter safety shield 100 also include second barrier 130, which can be a continuation of the first barrier or a separate barrier. If the barriers are separate, any space between them should be eliminated or minimized to prevent blood from passing through gaps between them. Alternatively, if a gap is not eliminated or minimized, it should be shaped to direct blood splatter away from the phlebotomist or user. When embodiments with second barrier 130 are attached to handheld blood tubing sealer 110, second barrier 130 can run substantially parallel to handheld blood tubing sealer 110 and above seal slot 140, which is used to seal blood tubing. Alternatively, the second barrier could create an obtuse or an acute angle with the first barrier. Accordingly, if blood tubing ruptures during sealing, blood splatter can be captured in a substantially horizontal direction by at least one barrier 120 and in a substantially vertical direction by second barrier 130. In certain embodiments, a third barrier or a third and fourth barrier can also be included.

Attachment mechanism 150 attaches at least one barrier 120 of blood splatter safety shield 100 to handheld blood tubing sealer 110. Blood splatter safety shield 100 can be permanently or removably attached to handheld blood tubing sealer 110. In some embodiments, blood splatter safety shield 100 is attached between seal slot 140 and seal lamp 160. Blood splatter safety shield 100 can also be attached in other positions that will separate a user from the location of sealing or heat-sealing of a segment of blood tubing so as to shelter the user from any blood exposure or splatter.

In some embodiments, attachment mechanism 150 comprises an opening in at least one barrier 120 through which handheld blood tubing sealer 110 can pass to expose seal slot 140 on one side of at least one barrier 120 with the rest of handheld blood tubing sealer 110 on the other side. Once in position, attachment mechanism 150 can be used to secure blood splatter safety shield 100 to handheld blood tubing sealer 110. Attachment mechanism 150 can also comprise any other means of attaching blood splatter safety shield 100 to handheld blood tubing sealer 110.

Blood splatter safety shield 100 is a simple, portable alternative to unwieldy face and/or eye shields typically used during blood tubing sealing. The blood splatter safety shield 100 allows for construction of inexpensive systems that can be modified and adapted for use with different blood tubing sealers and handheld blood tubing sealers. As shown in FIG. 1, in some embodiments of blood splatter safety shield 100, at least one barrier 120 can be transparent. This allows the user or phlebotomist to visualize sealing in seal slot 140 while handling handheld blood tubing sealer 110. In addition, blood splatter safety shield 100 can be constructed from transparent material including, without limitation, clear laminate, plastic, or glass.

FIG. 2 is a side view of the exemplary embodiment of blood splatter safety shield 200 depicted in FIG. 1. Blood splatter safety shield 200 is attached to handheld blood tubing sealer 210 via attachment mechanism 250 such that blood splatter safety shield 200 separates seal slot 240 on one side from seal lamp 260 and seal button 270 of handheld blood tubing sealer 210 on the other side. Accordingly, when a phlebotomist or user seals blood tubing in seal slot 240, blood splatter safety shield 200 will shelter the user from any blood splatter should the blood tubing rupture. As shown, blood splatter safety shield 200 has two barriers, at least one barrier 220 which is substantially perpendicular to handheld blood tubing sealer 210 and second barrier 230 which is substantially parallel to handheld blood tubing sealer 210 when blood splatter safety shield 200 is attached. However, some embodiments of blood splatter safety shield 200 have only at least one barrier 220.

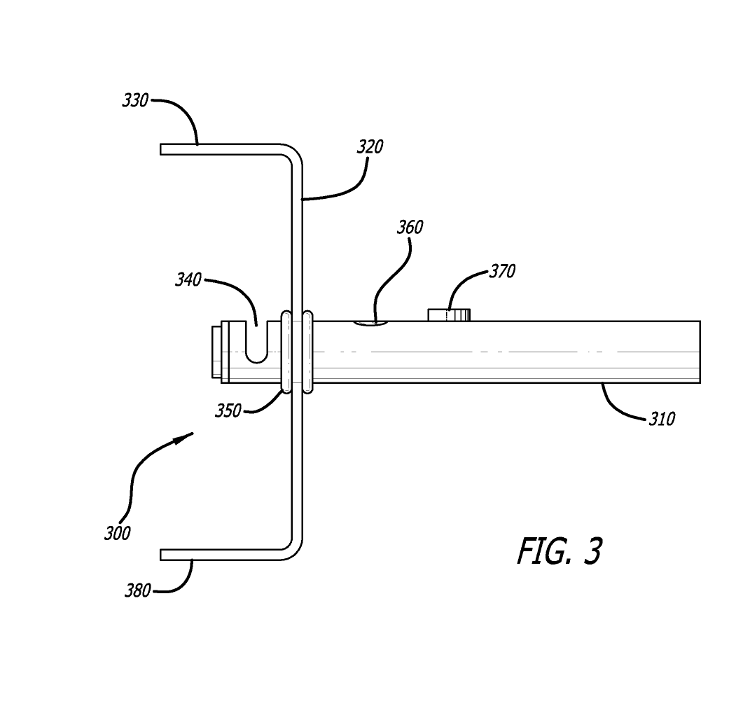

FIG. 3 is a side view of an alternative exemplary embodiment of blood splatter safety shield 300. Blood splatter safety shield 300 is attached to handheld blood tubing sealer 310 via attachment mechanism 350. Blood splatter safety shield 300 comprises at least one barrier 320, second barrier 330, and third barrier 380. When blood splatter safety shield 300 is attached to handheld blood tubing sealer 310, second barrier 330 and third barrier 380 can run substantially parallel to handheld blood tubing sealer 310. When attached, second barrier 330 can be located above seal slot 340 (or a similar sealing or heat sealing means of handheld blood tubing sealer 310), and third barrier 380 can be located below seal slot 340 (or a similar sealing or heat sealing means). In some embodiments, blood splatter safety shield 300 can be attached between seal slot 340 and seal lamp 360, such that a user as access to seal button 370 and seal lamp 360, but is separated from a segment of blood tubing being sealed at seal slot 340.

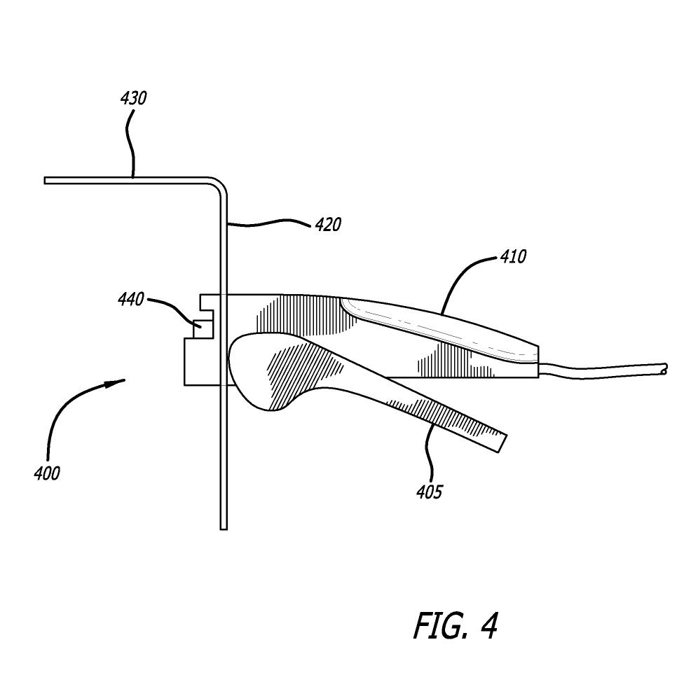

FIG. 4 illustrates yet another exemplary embodiment of blood splatter safety shield 400 attached to a different exemplary handheld blood tubing sealer 410. Handheld blood tubing sealer 410 comprises movable handle 405 and seal head 440, which is used to seal a segment of blood tubing. As shown, blood splatter safety shield 400 comprises at least one barrier 420 and second barrier 430. In one embodiment, at least one barrier 420 can be substantially perpendicular to handheld blood tubing sealer 410. In embodiments comprising second barrier 430 as well as at least one barrier 420, second barrier 430 can be substantially parallel to handheld blood tubing sealer 410. In some embodiments, blood splatter safety shield 400 can also comprise a third barrier. In other embodiments, blood splatter safety shield 400 can be substantially concave, substantially planar, substantially curved, or substantially convex.

Blood splatter safety shield 400 can be permanently or removably attached to handheld blood tubing sealer 410 between movable handle 405 and seal head 440. Accordingly, when a segment of blood tubing is sealed with seal head 440, blood splatter will be collected on blood splatter safety shield 400.

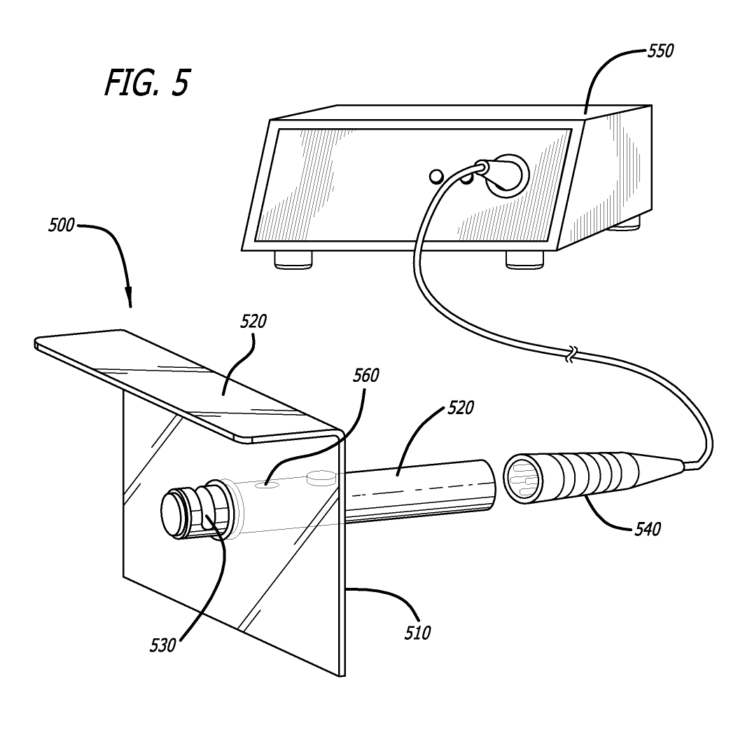

FIG. 5 illustrates exemplary blood tubing sealer 550 connected to its handheld blood tubing sealer 520 by coaxial cable 540. Blood tubing sealer 550 can be, without limitation, a Fenwal Autosealer Tubing Sealer, a Fresenius Kabi Composeal Mobilea tube sealer, or a similar battery-powered tubing sealer that has handheld blood tubing sealer 520 or a similar unit to which blood splatter safety shield 500 can attach. Blood splatter safety shield 500 has at least one barrier 510, and in some embodiments second barrier 520, to shelter a user from blood and blood-borne pathogens should a segment of blood tubing rupture before, during, or after it is sealed in seal slot 530 or a similar heat sealing head.

Utilization of blood splatter safety shield as described in this disclosure provides efficient, simple, and portable protection of users of handheld blood tubing sealers from blood-borne pathogens that may be transmitted via blood splatter before, during, or after sealing. Exemplary methods of the present description utilize blood splatter safety shields of the present description. For example, methods can include steps of: attaching blood splatter safety shield 500 to handheld blood tubing sealer 520; sealing a segment of blood tubing with handheld blood tubing sealer 520; and collecting blood splatter from the sealing step on at least one barrier 510 of blood splatter safety shield 500.

The attaching step of exemplary methods can include placing and securing handheld blood tubing sealer 520 through an opening in at least one barrier 510 such that blood splatter safety shield separates the user of handheld blood tubing sealer 520 from any blood exposed or splattered during sealing. Additionally, in some embodiments, exemplary seal slot 530 will be used in the sealing step. In certain embodiments of the collecting step, blood splatter can be collected on both at least one barrier 510 and on second barrier 520. Exemplary methods can also utilize a blood splatter safety shield attached to handheld blood tubing sealer 520, for example without limitation, between seal slot 530 and seal lamp 560, or in any other location to separate the user of handheld blood tubing sealer 520 from the sealing location of handheld blood tubing sealer 520.

Exemplary methods of the present description can include all of these steps or only some or one of these steps. In addition, exemplary methods can also include additional steps not fully described herein.

A wide variety of users, including without limitation phlebotomists, medical practitioners, and veterinary practitioners, will find that the systems and methods disclosed herein provide many advantages over existing safety precautions for blood extraction and blood tube sealing. The present disclosure provides methods and systems for user protection via a portable, inexpensive blood splatter safety shield that attaches directly or indirectly to a handheld blood tubing sealer to protect users from blood splatter without requiring the use of cumbersome, uncomfortable face or eye shields.

Unless otherwise indicated, all numbers expressing quantities and/or properties such as resolution and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the exemplary embodiments (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the exemplary embodiments and does not pose a limitation on the scope of the exemplary embodiments otherwise claimed. No language in the specification should be construed as indicating any non-claimed element essential to the practice of the exemplary embodiments.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the exemplary embodiments. Of course, variations on these described embodiments will become apparent to those of ordinary skill in the art upon reading the foregoing description. The inventor expects skilled artisans to employ such variations as appropriate, and the inventors intend for the embodiments to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Exemplary embodiments so claimed are inherently or expressly described and enabled herein.

In closing, it is to be understood that the exemplary embodiments disclosed herein are illustrative of the principles of the present disclosure. Other modifications that may be employed are within the scope of the disclosure. Thus, by way of example, but not of limitation, alternative configurations of the present exemplary embodiments may be utilized in accordance with the teachings herein. Accordingly, the present exemplary embodiments are not limited to that precisely as shown and described.

1. A blood splatter safety shield configured for attachment to a handheld blood tubing sealer comprising at least one barrier and an attachment mechanism to attach the at least one barrier to said handheld blood tubing sealer.

2. A blood splatter safety shield according to claim 1 wherein said handheld blood tubing sealer comprises a seal slot and a seal lamp and wherein said blood splatter safety shield is attached between the seal slot and the seal lamp of said handheld blood tubing sealer.

3. A blood splatter safety shield according to claim 1 wherein the at least one barrier runs substantially perpendicular to the handheld blood tubing sealer between a segment of blood tubing and a user of the handheld blood tubing sealer when the blood splatter safety shield is attached to the handheld blood tubing sealer.

4. A blood splatter safety shield according to claim 1 wherein said handheld blood tubing sealer is connected to a power source via a cord, is a self-contained unit with an internal power source, or is a self-contained unit charged via a charging unit.

5. A blood splatter safety shield according to claim 1 wherein the barrier is substantially planar and/or substantially transparent.

6. A blood splatter safety shield according to claim 1 further comprising a second barrier.

7. A blood splatter safety shield according to claim 6 wherein the second barrier runs substantially parallel to the handheld blood tubing sealer and is above the seal slot of the handheld blood tubing sealer when the blood splatter safety shield is attached to the handheld blood tubing sealer.

8. A blood splatter safety shield according to claim 6 wherein the second barrier is substantially planar and/or substantially transparent.

9. A blood splatter safety shield according to claim 1 wherein the attachment mechanism comprises an opening in the at least one barrier through which the handheld blood tubing sealer can pass and be secured in place.

10. A blood splatter safety shield according to claim 9 further comprising a gasket between the attachment mechanism and the handheld blood tubing sealer.

11. A system comprising a blood splatter safety shield and a handheld blood tubing sealer wherein the blood splatter safety shield is attached to the handheld blood tubing sealer and comprises at least one barrier.

12. A system according to claim 11 wherein the blood splatter safety shield is removably or permanently attached to the handheld blood tubing sealer.

13. A system according to claim 11 the splatter safety shield is attached to the handheld blood tubing sealer through an opening in the at least one barrier.

14. A system according to claim 13 further comprising a gasket at the opening.

15. A system according to claim 11 wherein during use the at least one barrier runs substantially perpendicular to the blood tubing sealer between blood tubing and a user of the blood tubing sealer.

16. A system according to claim 15 wherein the at least one barrier is substantially planar and/or substantially transparent.

17. A system according to claim 11 further comprising a second barrier.

18. A system according to claim 17 wherein said handheld blood tubing sealer comprises a seal slot and a seal lamp, and wherein, during use, the second barrier runs substantially parallel to the handheld blood tubing sealer and is above the seal slot of the handheld blood tubing sealer.

19. A system according to claim 17 wherein the second barrier is substantially planar and/or substantially transparent.

20. A method of preventing blood splatter from contacting a phlebotomist or user comprising attaching a blood splatter safety shield to a handheld blood tubing sealer.
